(19) United States
(12) Patent Application Publication
(10) Pub. No.: US 2017/0283693 A1
(43) Pub. Date: Oct. 5, 2017

(54) LUMINESCENT HALIDE PEROVSKITES, METHODS, COMPOSITIONS, AND DEVICES

(71) Applicant: Florida State University Research Foundation, Inc., Tallahassee, FL (US)

(72) Inventors: Biwu Ma, Tallahassee, FL (US); Zhao Yuan, Tallahassee, FL (US)

(21) Appl. No.: 15/476,174

(22) Filed: Mar. 31, 2017

Related U.S. Application Data
(60) Provisional application No. 62/316,090, filed on Mar. 31, 2016.

(51) Int. Cl. C09K 11/06; H01L 33/50; C07C 213/08; C09K 11/02; C07C 217/42

(52) U.S. Cl. CPC C09K 11/06; C09K 11/025; C07C 217/42; C07C 213/08; H01L 33/502; C09K 2211/188; C09K 2211/10; H01L 2933/0041

(57) ABSTRACT

Provided herein are metal halide perovskites, which may have a layered structure. The metal halide perovskites may emit white light. Also provided herein are neat films of the metal halide perovskites, films comprising a polymer matrix and the metal halide perovskites, and devices that include the metal halide perovskites. Methods of making metal halide perovskites also are provided.

### CROSS-REFERENCE TO RELATED APPLICATIONS

This application claims priority to U.S. Provisional Patent Application No. 62/316,090, filed Mar. 31, 2016, which is incorporated herein by reference.

### BACKGROUND

Solid state lighting (SSL) devices typically have higher power efficiencies than conventional incandescent and fluorescent lamps. To generate white emission, typical SSL devices rely on light emitting diodes (LEDs) coated with a single phosphor, such as a GaN-based blue LED chip coated with a YAG:Ce yellow-emitting phosphor, or a mixture of phosphors, such as an ultraviolet (UV) LED coated with blue, green, and red phosphors. Both of these approaches, however, typically suffer from one or more disadvantages.

For example, single yellow/red phosphor coated LEDs typically emit white light with poor color rendition, due at least in part to discontinuities in the phosphor's emission. Also, mixing phosphors often results in efficiency losses due to self-absorption, and the different degradation rates of the individual phosphors can lead to changes in emission color over time.

Moreover, most known white emitting phosphors include primarily inorganic materials, which typically require emissive dopants or surface sites. The quantum efficiencies of these white emitting phosphors usually are relatively low, and their production can be complex and/or expensive. To date, there is no commercially available SSL devices that rely on single component white emitting phosphors.

There remains a need for phosphors that are single source broadband white light emitters, highly luminescent, efficient, easy to produce, stable, long lasting, inexpensive, and/or easily dispersed due to favorable crystallization kinetics.

### BRIEF SUMMARY

Provided herein are metal halide perovskites that address one or more of the foregoing needs, and may be used as a single source white light emitting phosphor.

In embodiments, the metal halide perovskites comprise a crystal having a unit cell according to formula (I):

(A-R—B)MX₄ (I);

wherein the crystal has a layered structure; M is a metal atom selected from Pb, Sn, Sb, Cu, Ge, Mn, Co, Bi, Ni, Zn, or Eu; X is a halide ion selected from Cl, Br, or I; and (A-R—B) is a ligand in which (i) A and B are independently selected from a primary ammonium cation, a secondary ammonium cation, or a tertiary ammonium cation, and (ii) R comprises a moiety of 4 to 12 atomic units, wherein each atomic unit comprises a carbon atom, a sulfur atom, or an oxygen atom.

Also provided herein are films comprising a metal halide perovskite. In one embodiment, the films are neat films of a metal halide perovskite of formula (I). In another embodiment, the films comprise a metal halide perovskite of formula (I), and a polymer matrix. The metal halide perovskite may be dispersed in the polymer matrix, and/or present in the polymer matrix at an amount of about 1% to about 25% by weight of the film.

Also provided are methods of making metal halide perovskites, including metal halide perovskites according to formula (I). In embodiments, the methods comprise contacting a metal halide and an organic acid in a non-polar organic liquid; and adding a ligand to the non-polar organic liquid; wherein the ligand comprises a compound according to the following formula:

(A′-R—B′),

wherein A′ and B′ are independently selected from a primary amine, a secondary amine, or a tertiary amine, and R comprises a moiety of 4 to 12 atomic units, wherein each atomic unit comprises a carbon atom, a sulfur atom, or an oxygen atom; and the metal halide is selected from lead(II) halide, tin(II) halide, antimony(III) halide, copper(II) halide, germanium(II) halide, manganese(II) halide, cobalt(II) halide, bismuth(III) halide, nickel(II) halide, zinc(II) halide, or europium(III) halide.

### BRIEF DESCRIPTION OF THE DRAWINGS

FIG. 1 depicts one embodiment of a metal halide perovskite having a layered structure.

FIG. 2A includes a transmission electron microscopy image, and the electron diffraction pattern (inset) of one embodiment of a metal halide perovskite.

FIG. 2B depicts a powder X-ray diffraction pattern and a simulated pattern from room-temperature single-crystal data of one embodiment of a metal halide perovskite.

FIG. 2C is an atomic force microscopy image of one embodiment of a metal halide perovskite.

FIG. 2D is a height profile corresponding to the diagonal line traversing FIG. 2C.

FIG. 3A depicts the absorption and photoluminescence spectra of an as-prepared embodiment of a metal halide perovskite in toluene, and in the form of a thin film.

FIG. 3B depicts the time-resolved photoluminescence decays of a neat thin film of one embodiment of a metal halide perovskite.

FIG. 4 depicts the photoluminescence spectrum of one embodiment of a metal halide perovskite when embedded in an embodiment of a polymeric matrix.

FIG. 5 depicts the emission spectra of one embodiment of a white light-emitting device at different operating voltages.

FIG. 6 depicts the photoluminescence intensity of one embodiment of a light-emitting device operated at room temperature.

### DETAILED DESCRIPTION

Provided herein are metal halide perovskites having a layered crystal structure. The metal halide perovskites may be employed as a single source white light emitting phosphor. In one embodiment, the metal halide perovskites are highly luminescent layered metal halide perovskites, such as (EDBE)PbBr₄, with emission across the entire visible spectrum. The metal halide perovskites provided herein may be made with a facile room temperature one-pot synthesis, with yields that may be as high as about 96%. The metal halide perovskites provided herein also may be used as a single component down conversion phosphor in UV-pumped white light emitting devices. The metal halide perovskites provided herein also may be substantially stable in liquid processed neat thin films and/or doped polymer thin films.

In embodiments, the metal halide perovskites comprise a crystal having a unit cell according to formula (I):

(A-R—B)MX₄ (I);

wherein the crystal has a layered structure; M is a metal atom selected from Pb, Sn, Sb, Cu, Ge, Mn, Co, Bi, Ni, Zn, or Eu; X is a halide ion selected from Cl, Br, or I; and (A-R—B) is a ligand in which (i) A and B are independently selected from a primary ammonium cation, a secondary ammonium cation, or a tertiary ammonium cation, and (ii) R comprises a moiety of 4 to 12 atomic units, wherein each atomic unit comprises a carbon atom, a sulfur atom, or an oxygen atom. The metal halide perovskites of formula (I) may emit white light.

In one embodiment, M is Pb, X is Br, and the ligand is 2,2′-(ethane-1,2-diylbis(oxy))diethanaminium (EDBE), and the metal halide perovskite has a unit cell of the following formula:

(EDBE)PbBr₄.

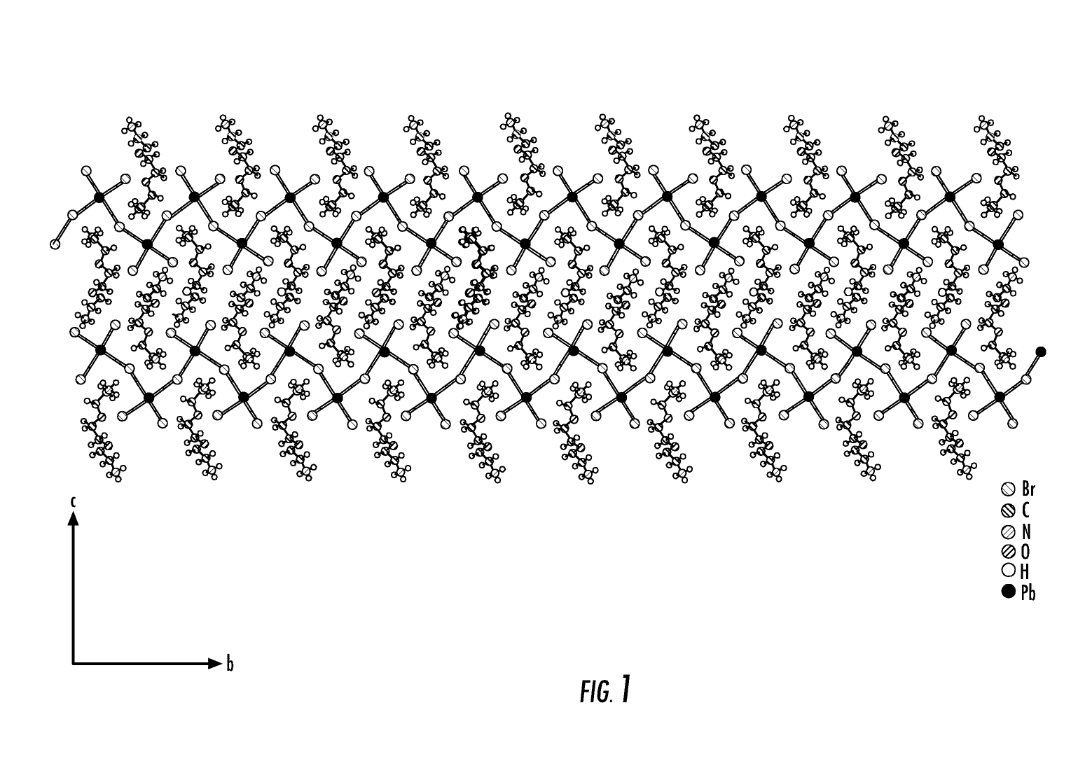

The metal halide perovskite having the unit cell of formula (EDBE)PbBr₄ may have a layered crystal structure corresponding to the ball-and-stick configuration of FIG. 1.

As used herein, the phrases “layered structure”, “layered crystal structure”, or “corrugated structure” refer to crystal structures that include at least two rows of adjacent unit cells (when viewed along axis “a”, as in FIG. 1), wherein the metal atoms and halide atoms of a first row of unit cells are separated from the metal atoms and halide atoms of an adjacent second row of unit cells by an arrangement of ligands. One embodiment of such a “layered structure” is depicted at FIG. 1, which includes two horizontal rows of metal atoms and halide atoms, and the metal atoms and halide atoms of each row are isolated from each other by the EDBE ligands depicted in the middle horizontal row.

Not wishing to be bound by any particular theory, it is believed that the layered structure of embodiments of the metal halide perovskites provided herein imparts the metal halide perovskites with crystallization kinetics that are more favorable that those of known perovskites.

### Ligands

The ligands of the metal halide perovskites provided herein generally may be any compound that includes at least two ammonium groups, and is capable of forming the layered crystal structures provided herein. The at least two ammonium groups may be terminal groups. The ammonium groups may be primary ammonium groups, secondary ammonium groups, tertiary ammonium groups, or a combination thereof. In addition to at least two ammonium groups, the ligands may include at least one hydrocarbyl group, at least one atom capable of participating in hydrogen bonding, or a combination thereof.

In embodiments, the ligand of the unit cells of the metal halide perovskites has a structure according to the following formula:

(A-R—B),

wherein (i) A and B are independently selected from a primary ammonium cation, a secondary ammonium cation, or a tertiary ammonium cation, and (ii) R comprises a moiety of 4 to 12 atomic units, wherein each atomic unit comprises a carbon atom, a sulfur atom, or an oxygen atom. The 4 to 12 atomic units are covalently bonded together, one of the 4 to 12 atomic units is covalently bonded to A, and one of the 4 to 12 atomic units is covalently bonded to B. The 4 to 12 atomic units may form a branched structure or an unbranched structure. The unbranched structure may be a substantially linear moiety. Non-limiting examples of the atomic units that may be selected for “R” include a thioether (—S—), a disulfide (—S—S—), an ether (O), a tetravalent carbon (═C═ or —C≡), a trivalent carbon (—CH═), and a divalent carbon (—CH₂—). Due to the fact that A and B may represent 1°, 2°, or 3° ammonium cations, the dashed lines in the formula (A-R—B) and the other formulas herein may represent a single bond, a double bond, or a triple bond.

In embodiments, the ligand has a structure according to formula (A-R—B), wherein A and B are primary ammonium cations, and the ligand is a compound of the following formula:

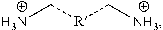

wherein R′ comprises an unbranched moiety of 2 to 10 atomic units selected independently from S, O, C, CH, or CH₂. In one embodiment, R′ comprises 4 to 8 atomic units selected independently from S, O, or CH₂, and the ligand is a compound of the following structure:

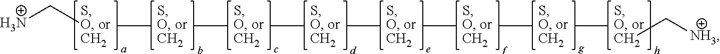

wherein a, b, c, and d are 1, and e, f, g, and h are selected independently from 0 or 1. In a particular embodiment, a, b, c, d, e, and f are 1, g and h are 0, and the ligand has the following structure:

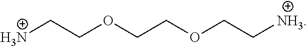

In another embodiment, a, b, c, d, e, and f are 1, g and h are 0, and the ligand has the following structure:

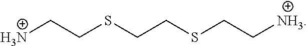

In embodiments, the ligand has a structure according to formula (A-R—B), wherein A and B are secondary ammonium cations, and the ligand is a compound of the following formula:

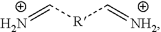

wherein R′ comprises an unbranched moiety of 2 to 10 atomic units selected independently from S, O, C, CH, or CH₂. In one embodiment, R′ comprises 4 to 8 atomic units selected independently from S, O, or CH₂, and the ligand is a compound of the following structure:

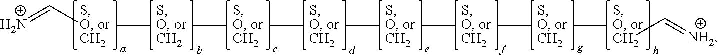

wherein a, b, c, and d are 1, and e, f, g, and h are selected independently from 0 or 1. In a particular embodiment, a, b, c, d, e, and f are 1, g and h are 0, and the ligand has the following structure:

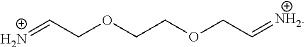

In another embodiment, a, b, c, d, e, and f are 1, g and h are 0, and the ligand has the following structure:

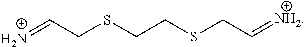

In embodiments, the ligand has a structure according to formula (A-R—B), wherein A and B are tertiary ammonium cations, and the ligand is a compound of the following formula:

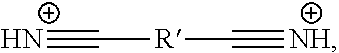

wherein R′ comprises an unbranched moiety of 2 to 10 atomic units selected independently from S, O, C, CH, or CH₂. In one embodiment, R′ comprises 4 to 8 atomic units selected independently from S, O, or CH₂, and the ligand is a compound of the following structure:

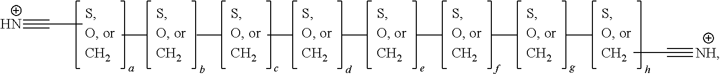

wherein a, b, c, and d are 1, and e, f, g, and h are selected independently from 0 or 1. In a particular embodiment, a, b, c, d, e, and f are 1, g and h are 0, and the ligand has the following structure:

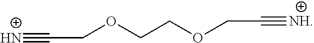

In another embodiment, a, b, c, d, e, and f are 1, g and h are 0, and the ligand has the following structure:

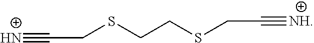

Other ligands are envisioned, including, for example, ligands in which one or more of the ammonium groups is not a terminal ammonium group, and/or ligands that include at least one oxygen atom and at least one sulfur atom.

### Metal Atoms and Halide Ions

The metal atoms of the unit cells of the metal halide perovskites provided herein may be any metal atom capable of forming a metal halide. In embodiments, the metal atom is selected from Pb, Sn, Sb, Cu, Ge, Mn, Co, Bi, Ni, Zn, or Eu. In one embodiment, the metal atom is selected from Pb, Sn, Cu, Ge, Mn, Co, Ni, or Zn. In a particular embodiment, the metal atom is lead.

The halide atoms of the unit cells of the metal halide perovskites provided herein may be selected from chloride, bromide, or iodide. In embodiments, the metal atom is selected from Pb, Sn, Sb, Cu, Ge, Mn, Co, Bi, Ni, Zn, or Eu, and the halide ion is bromide. In one embodiment, the metal atom is selected from Pb, Sn, Cu, Ge, Mn, Co, Ni, or Zn, and the halide ion is bromide. In a particular embodiment, the metal atom is lead, and the halide ion is bromide.

In embodiments, the metal atom is selected from Pb, Sn, Sb, Cu, Ge, Mn, Co, Bi, Ni, Zn, or Eu, and the halide ion is chloride. In one embodiment, the metal atom is selected from Pb, Sn, Cu, Ge, Mn, Co, Ni, or Zn, and the halide ion is chloride. In a particular embodiment, the metal atom is lead, and the halide ion is chloride.

In embodiments, the metal atom is selected from Pb, Sn, Sb, Cu, Ge, Mn, Co, Bi, Ni, Zn, or Eu, and the halide ion is iodide. In one embodiment, the metal atom is selected from Pb, Sn, Cu, Ge, Mn, Co, Ni, or Zn, and the halide ion is iodide. In a particular embodiment, the metal atom is lead, and the halide ion is iodide.

### Films and Devices

Films comprising one or more metal halide perovskites also are provided herein. In embodiment, the film is a neat film of a metal halide perovskite. The neat film may be made by casting. For example, the neat film of a metal halide perovskite may be made by drop casting a liquid comprising an amount of the metal halide perovskite. The phrase “neat film”, as used herein, refers to a film containing at least 95%, by weight, of a metal halide perovskite. The neat films may include up to 5%, by weight, of residual liquids, impurities, etc.

In embodiments, the films provided herein comprise a metal halide perovskite of formula (I), and a polymer matrix, wherein the metal halide perovskite is dispersed in the polymer matrix. The metal halide perovskite may be present at an amount of about 1% to about 25% by weight of the film. The metal halide perovskite may be at least substantially evenly dispersed in the polymer matrix, or the metal halide perovskite may be unevenly dispersed in the polymer matrix. In one embodiment, the metal halide perovskite is present at an amount of about 5% to about 15% by weight of the film. In another embodiment, the metal halide perovskite is present at an amount of about 10% by weight of the film. The polymer matrix may comprise any one or more polymers capable of hosting a dispersion of the metal halide perovskite. In one embodiment, the polymer matrix comprises polystyrene. The films may emit white light, and/or the PLQE of the film may be at least 15%. In a particular embodiment, the PLQE is about 18%.

Devices also are provided herein. The metal halide perovskites may emit light when employed in the devices. In embodiments, the devices comprise a metal halide perovskite of formula (I), wherein the metal halide perovskite is a phosphor in the device. The device may emit white light, due at least in part to the emission of the metal halide perovskite. In the devices, the metal halide perovskites may be in the form of a neat film, a layer comprising a polymer matrix, or a combination thereof.

The devices herein may be light emitting diodes. Therefore, the devices provided herein may include an electrode, a counterelectrode, at least one light emitting layer comprising a metal halide perovskite, and one or more of the following layers: electron transport layer, hole transport layer, electron blocking layer, and hole blocking layer. The electron transport layer, hole transport layer, electron blocking layer, and hole blocking layer, independently of each other, may be doped or undoped.

### Methods of Making Metal Halide Perovskites

Provided herein are methods of making metal halide perovskites, including metal halide perovskites according to formula (I).

In embodiments, the methods comprise contacting a metal halide and an organic acid in a non-polar organic liquid; and adding a ligand precursor to the non-polar organic liquid.

The steps of the methods of making metal halide perovskites may be performed at any temperature capable of facilitating the formation of the metal halide perovskites, including the layered crystal structure. Typically, the temperature is less than the boiling point of the non-polar liquid in which the metal halide and organic acid are contacted. The steps may be performed at temperatures of about 0° C. to about 75° C. At least one of the steps of the methods of making metal halide perovskites may be performed at room, i.e., ambient, temperature and/or pressure. In embodiments, at least one of the contacting of the metal halide and the organic acid in the non-polar organic liquid, and the adding of the ligand to the non-polar organic liquid occur at ambient temperature. In one embodiment, both the contacting of the metal halide and the organic acid in the non-polar organic liquid, and the adding of the ligand to the non-polar organic liquid occur at ambient temperature. Typically, room temperature is about 20° C. to about 26° C.

Due at least in part to the low cost of raw materials, room temperature synthesis, and/or facile solution processing, the metal halide perovskites provided herein may permit SSL based on inexpensive and/or large-area coatings. Moreover, due to the fact that the metal halide perovskites provided herein may have a PLQE of at least 15%, and/or be produced with methods that permit exceptional solution-processability at low-temperature, accurate stoichiometry control, and/or high yield, the metal halide perovskites provided herein may represent, at least in some embodiments, an alternative to conventional inorganic rare earth based phosphors and/or quantum-dot-based phosphors that currently are used in white LEDs.

The methods of making metal halide perovskites provided herein also may include separating the metal halide perovskites from the non-polar organic liquid. The separation may occur by any technique known in the art, including, but not limited to, centrifugation, filtration, etc.

The non-polar liquid may be a single liquid or a combination of two or more liquids. Non-limiting examples of suitable non-polar liquids include aromatic hydrocarbons, aliphatic hydrocarbons, and alicyclic hydrocarbons. Non-limiting examples of aromatic hydrocarbons include benzene, toluene, xylene, ethylbenzene, tetralin, etc. Non-limiting examples of aliphatic hydrocarbons include butane, pentane, isopentane, hexane, isohexane, heptane, octane, isooctane, naphtha, gasoline, kerosene, mineral oil, etc. Non-limiting examples of alicyclic hydrocarbons include cyclopentane, cyclohexane, methylcyclopentane, Decalin, indane, etc.

In embodiments, the metal halide is selected from lead(II) halide, tin(II) halide, antimony(III) halide, copper(II) halide, germanium(II) halide, manganese(II) halide, cobalt(II) halide, bismuth(III) halide, nickel(II) halide, zinc(II) halide, or europium(III) halide. In one embodiment, the metal halide is selected from lead(II) halide, tin(II) halide, copper(II) halide, germanium(II) halide, manganese(II) halide, cobalt(II) halide, nickel(II) halide, or zinc(II) halide. In a particular embodiment, the metal halide is lead(II) halide.

In embodiments, the metal chloride is selected from lead(II) chloride, tin(II) chloride, antimony(III) chloride, copper(II) chloride, germanium(II) chloride, manganese(II) chloride, cobalt(II) chloride, bismuth(III) chloride, nickel(II) chloride, zinc(II) chloride, or europium(III) chloride. In one embodiment, the metal chloride is selected from lead(II) chloride, tin(II) chloride, copper(II) chloride, germanium(II) chloride, manganese(II) chloride, cobalt(II) chloride, nickel(II) chloride, or zinc(II) chloride. In a particular embodiment, the metal chloride is lead(II) chloride.

In embodiments, the metal bromide is selected from lead(II) bromide, tin(II) bromide, antimony(III) bromide, copper(II) bromide, germanium(II) bromide, manganese(II) bromide, cobalt(II) bromide, bismuth(III) bromide, nickel(II) bromide, zinc(II) bromide, or europium(III) bromide. In one embodiment, the metal bromide is selected from lead(II) bromide, tin(II) bromide, copper(II) bromide, germanium(II) bromide, manganese(II) bromide, cobalt(II) bromide, nickel(II) bromide, or zinc(II) bromide. In a particular embodiment, the metal bromide is lead(II) bromide.

In embodiments, the metal iodide is selected from lead(II) iodide, tin(II) iodide, antimony(III) iodide, copper(II) iodide, germanium(II) iodide, manganese(II) iodide, cobalt(II) iodide, bismuth(III) iodide, nickel(II) iodide, zinc(II) iodide, or europium(III) iodide. In one embodiment, the metal iodide is selected from lead(II) iodide, tin(II) iodide, copper(II) iodide, germanium(II) iodide, manganese(II) iodide, cobalt(II) iodide, nickel(II) iodide, or zinc(II) iodide. In a particular embodiment, the metal iodide is lead(II) iodide.

### Organic Acid

Generally, any organic acid capable of protonating an amine may be used in the methods provided herein. Not wishing to be bound by any particular theory, it is believed that the organic acid may protonate the amines of a ligand, at least assist in solubilizing at least a portion of the metal halide, and/or stabilize the resulting perovskites.

The organic acid used in the methods provided herein may comprise a carboxylic acid functional group. The carboxylic acid functional group may be substituted with a linear or branched monovalent hydrocarbyl group that may be unsubstituted or substituted.

In embodiments, the organic acid used in the methods provided herein comprises a compound according to formula (III):

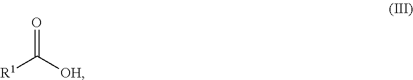

wherein R¹ is a monovalent C₅-C₂₀ hydrocarbyl. In one embodiment, the organic acid used in the methods provided herein comprises a compound according to formula (III), wherein R₁ is a monovalent C₅-C₉ hydrocarbyl. In another embodiment, the organic acid used in the methods provided herein comprises a compound according to formula (III), wherein R₁ is an unsubstituted, unbranched, and monovalent C₇ hydrocarbyl, and the organic acid is octanoic acid:

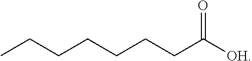

The amount of organic acid used in the methods provided herein may be an amount sufficient to protonate the amines of the ligand precursor. For example, when the ligand precursor includes two amines, at least a 2:1 molar ratio of organic acid to the ligand precursor may be used in the methods provided herein.

### Ligand Precursors

Generally, the ligand precursor of the methods of making metal halide perovskites may be any compound capable of forming a ligand described herein.

The ligand precursor of the methods of making metal halide perovskites may comprise a compound according to the following formula:

(A′-R—B′),

wherein A′ and B′ are independently selected from a primary amine, a secondary amine, or a tertiary amine, and R comprises a moiety of 4 to 12 atomic units, wherein each atomic unit comprises a carbon atom, a sulfur atom, or an oxygen atom. Therefore, the R group of the ligand precursor may correspond to the R group of the ligands herein, and the A′ and B′ groups of the ligand precursor may be unprotonated amines that, upon protonation, form the A and B ammonium groups, respectively, of the ligands herein.

In embodiments, the ligand precursor has a structure according to formula (A′-R—B′), wherein A′ and B′ are primary amines, and the ligand precursor is a compound of the following formula:

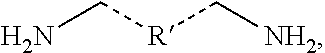

wherein R′ comprises an unbranched moiety of 2 to 10 atomic units selected independently from S, O, C, CH, or CH₂. In one embodiment, R′ comprises 4 to 8 atomic units selected independently from S, O, or CH₂, and the ligand precursor is a compound of the following structure:

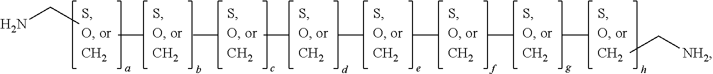

wherein a, b, c, and d are 1, and e, f, g, and h are selected independently from 0 or 1. In a particular embodiment, a, b, c, d, e, and f are 1, g and h are 0, and the ligand precursor has the following structure:

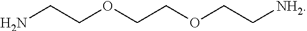

In another embodiment, a, b, c, d, e, and f are 1, g and h are 0, and the ligand precursor has the following structure:

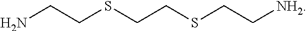

In embodiments, the ligand precursor has a structure according to formula (A′-R—B′), wherein A′ and B′ are secondary amines, and the ligand precursor is a compound of the following formula:

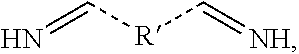

wherein R′ comprises an unbranched moiety of 2 to 10 atomic units selected independently from S, O, C, CH, or CH₂. In one embodiment, R′ comprises 4 to 8 atomic units selected independently from S, O, or CH₂, and the ligand precursor is a compound of the following structure:

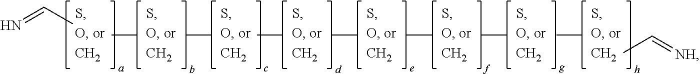

wherein a, b, c, and d are 1, and e, f, g, and h are selected independently from 0 or 1. In a particular embodiment, a, b, c, d, e, and f are 1, g and h are 0, and the ligand precursor has the following structure:

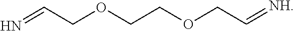

In another embodiment, a, b, c, d, e, and f are 1, g and h are 0, and the ligand precursor has the following structure:

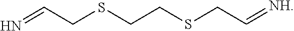

In embodiments, the ligand has a structure according to formula (A′-R—B′), wherein A′ and B′ are tertiary amines, and the ligand precursor is a compound of the following formula:

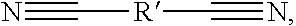

wherein R′ comprises an unbranched moiety of 2 to 10 atomic units selected independently from S, O, C, CH, or CH₂. In one embodiment, R′ comprises 4 to 8 atomic units selected independently from S, O, or CH₂, and the ligand precursor is a compound of the following structure:

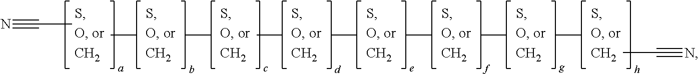

wherein a, b, c, and d are 1, and e, f, g, and h are selected independently from 0 or 1. In a particular embodiment, a, b, c, d, e, and f are 1, g and h are 0, and the ligand precursor has the following structure:

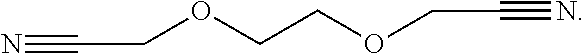

In another embodiment, a, b, c, d, e, and f are 1, g and h are 0, and the ligand precursor has the following structure:

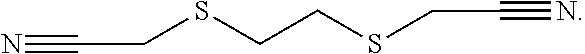

Other ligand precursors are envisioned, including, for example, ligand precursors in which one or more of the amino groups is not a terminal amino group, and/or ligand precursors that include at least one sulfur atom and at least one oxygen atom.

The phrases “C₅-C₂₀ hydrocarbyl”, “C₅-C₉ hydrocarbyl, “C₇ hydrocarbyl,”and the like, as used herein, generally refer to aliphatic, aryl, or arylalkyl groups containing 5 to 20, 5 to 9, or 7 carbon atoms. Examples of aliphatic groups, in each instance, include, but are not limited to, an alkyl group, a cycloalkyl group, an alkenyl group, a cycloalkenyl group, an alkynyl group, an alkadienyl group, a cyclic group, and the like, and includes all substituted, unsubstituted, branched, and linear analogs or derivatives thereof, in each instance having 5 to about 20 carbon atoms, 5 to 9 carbon atoms, 7 carbon atoms, etc. Examples of alkyl groups include, but are not limited to, methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). Representative alkenyl moieties include vinyl, allyl, 1-butenyl, 2-butenyl, isobutylenyl, 1-pentenyl, 2-pentenyl, 3-methyl-l-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl. Examples of aryl or arylalkyl moieties include, but are not limited to, anthracenyl, azulenyl, biphenyl, fluorenyl, indan, indenyl, naphthyl, phenanthrenyl, phenyl, 1,2,3,4-tetrahydro-naphthalene, tolyl, xylyl, mesityl, benzyl, and the like, including any heteroatom substituted derivative thereof.

Unless otherwise indicated, the term “substituted,” when used to describe a chemical structure or moiety, refers to a derivative of that structure or moiety wherein one or more of its hydrogen atoms is substituted with a chemical moiety or functional group such as alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), tertiary amine (such as alkylamino, arylamino, arylalkylamino), aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) or urea (—NHCONH-alkyl-).

It must be noted that, as used in the written description and the appended claims, the singular forms “a,” “an,” and “the” include plural referents unless the context clearly dictates otherwise. Thus, for example, reference to “a non-polar liquid” includes mixtures of non-polar liquids, and the like.

### EXAMPLES

The present invention is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein.

For the following examples, lead(II) bromide (99.999%), 2,2′-(ethylenedioxy)bis (ethylamine) (98%), octanoic acid (98%) and hexane (98.5%, mixture of isomers) were purchased from Sigma-Aldrich. All reagents and liquids were used without further purification unless otherwise stated. Spectroscopic grade liquids were used in the UV-Vis and photoluminescence spectroscopic measurements.

### EXAMPLE 1

### Synthesis of (EDBE)PbBr₄ Microscale Perovskites

(EDBE)PbBr₄ microscale perovskites were prepared using a one-pot, polar liquid-free synthetic method.

Lead(II) bromide (0.1 mmol)(36.7 mg) was added to a 5 mL hexane solution that included 1.0 mmol octanoic acid (160 μL), followed by the injection of 0.3 mmol 2,2′-(ethylenedioxy)bis(ethylamine) (44 μL) to form a turbid solution.

The reaction solution was stirred at room temperature for 24 hours until a white dispersed solution was formed. The white microscale perovskites then were obtained by centrifugation to remove the unreacted materials in the clear supernatant, affording a white powder in a 96% yield after drying under vacuum (see Table 1).

Organic acids other than octanoic acid were used in similar procedures, including hexanoic acid and stearic acid, but the below-described data and characterizations were performed on the microscale perovskites obtained from the foregoing method that used octanoic acid.

### EXAMPLE 2

### Characterization of the (EDBE)PbBr₄ Microscale Perovskites

The microscale lead(II) bromide perovskite of Example 1 was fully characterized by Transmission Electron Microscopy (TEM), X-ray Powder Diffraction (XRPD), Atomic Force Microscopy (AFM), Proton Nuclear Magnetic Resonance (¹H NMR), and Thermogravimetric Analysis (TGA).

These analyses were believed to reveal that the perovskite of Example 1 was produced at a yield of about 96%, and had a layered structure.

The as-prepared perovskite of Example 1 was dispersible in non-polar organic liquids, such as toluene. When the perovskite was dispersed, a stable dispersion was formed. The dispersibility and/or formation of the stable dispersed solution was believed to be the result of improved crystallization kinetics imparted by the layer structure of the perovskite of Example 1.

Nuclear magnetic resonance (¹H-NMR): ¹H NMR spectra were acquired at room temperature on Bruker AVANCE III NMR Spectrometers with a 500 MHz Bruker magnet. All chemical shifts (δ) were reported in ppm relative to tetramethylsilane (TMS).

Only one set of peaks from 2,2′-(ethylenedioxy)bis(ethylammonium) was observed in the proton NMR spectrum, which was believed to suggest that the octanoic acid had been completely removed from the products with the liquid.

Thermogravimetry analysis (TGA): TGA was carried out using a TA instruments Q50 TGA system. The samples were heated from room temperature (˜22° C.) to 800° C. at a rate of 5 ° C·min⁻¹, under a nitrogen flux of 100 mL·min⁻¹.

TGA analysis was also used to characterize the composition and purity of the microscale perovskite of Example 1 . Two notable weight changes were observed: the first was believed to correspond to the loss of the organic bisammonium bromide of 46% at 286° C.; and the other was believed to be related to the release of the inorganic lead bromide of 54% at 513° C. Based on these TGA results, the weight ratios between organic and inorganic salts were calculated to be 0.85, which was believed to suggest an approximately 1:1 molar ratio between the organic and inorganic components, which is typically a feature of corrugated 2D layered perovskite structures.

TABLE 1
Chemical yield and composition of the component molar ratio in
(EDBE)PbBr₄ microscale perovskites of Example 1 according to TGA
and ¹H NMR data.

| | | Starting | Molar | Theoretical | |
|---|---|---|---|---|---|
| Product | Reagent | amount | ratio^{a} | ratio^{b} | Yield |
| Microscale | Octanoic | 1.00 mmol | 0.00 | 0.00 | 96% |
| Perovskites | Acid | | | | |
| | EDBE | 0.30 mmol | 1.01 | 1.00 | |
| | PbBr₂ | 0.10 mmol | 1.00 | 1.00 | |

^{a}Based on ¹H NMR and TGA data;
^{b}based on the chemical formula: (EDBE)PbBr₄.

The thermo- and photo-stability of the phosphor can be important, especially when the phosphor is used in LEDs, including high power LEDs. The microscale perovskites of Example 1 exhibited great thermo- and photo-stability: the thermal decomposition temperature was measured as 260° C., which was believed to demonstrate the high thermo-stability of the microscale perovskites. Also, the dried microscale perovskites could be kept in the solid state under an air atmosphere for at least about two weeks without any change of its PLQE.

The photoluminescence intensity decreased upon increasing the temperature, which was believed to be common for light emitting materials as a result of enhanced nonradiative decay. When the temperature was returned to room temperature from 80° C., the emission recovered, which was believed to demonstrate that the microscale perovskites of Example 1 had a very favorable thermo-stability. Moreover, no change in emission color or intensity of the microscale perovskite neat thin films was observed after 120 min of UV irradiation (365 nm, 4 W, 550 μW/cm²). The favorable thermo- and/or photo-stability was believed to suggest that the microscale perovskites herein can be used succesfully in down-conversion white light emitting devices.

Transmission Electron Microscopy images (TEM): Microstructural characterization was performed using transmission electron microscopy, on a cold field emission JEOL JEM-ARM200cF at 200kV. Low intensity illumination and fast acquisition time were used during data collection to avoid beam damage. TEM samples were prepared by depositing a few drops of the perovskite solution on a carbon film-supported copper grid (200 mesh); the samples were subsequently dried overnight.

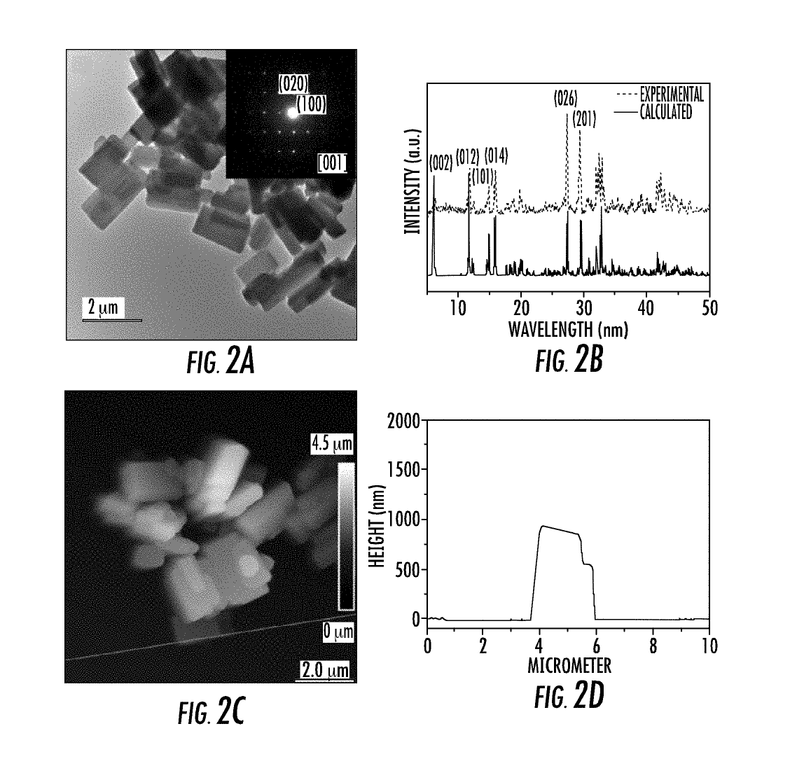

FIG. 2A shows the TEM images of the as-prepared microscale lead(II) bromide perovskites of Example 1 with their electron diffraction patterns. Rectangular-shaped single crystals with a lateral dimension size of 1˜3 micrometers were observed. The electron diffraction pattern in the inset of FIG. 2A depicts the [001] orientation electron diffraction from the individual microscale perovskite, which suggested a single crystalline structure.

Unlike typical 2D and 3D perovskite structures, the perovskite of Example 1 adopted a <110>-oriented hybrid structure as shown at FIG. 1. FIG. 1 is a ball-and-stick depiction of the layered crystal structure of the perovskite of Example 1 when viewed along the a axis. The hydrogen bonds between terminal ammonium groups and oxygens in the bisammonium ligands appeared to enforce a paired configuration, which was believed to be at least partially responsible for stabilizing the <110>structure. With a monoclinic crystal system, these microscale perovskites were believed to have different d-spacings along three axes. As shown at FIG. 2A, the d-spacings measured from the electron diffraction patterns was 6.09±0.1 Å of the (100) plane and 8.98 ±0.1 A of the (010) plane, which was believed to be consistent with the values of their single crystalline bulk samples.

X-ray powder diffraction (XRPD): The XRD analysis was performed on Panalytical X′PERT Pro Powder X-Ray Diffractometer using Copper X-ray tube (standard) radiation at a voltage of 40 kV and 40 mA, and X'Celerator RTMS detector. The diffraction pattern was scanned over the angular range of 5-50 degree (20) with a step size of 0.02, at room temperature.

FIG. 2B depicts the XRD pattern for the unoriented microscale perovskites of Example 1, presenting several feature peaks from the single crystal X-ray structure. The excellent fit to the simulated pattern was believed to confirm the crystalline structure of the microscale perovskites of Example 1.

Atomic Force Microscopy images (AFM): AFM measurements were conducted using a Bruker Icon instrument. All measurements were performed in the standard tapping mode with OTESPA-R3 tips from Bruker.

AFM was used to further characterize the morphological properties of the microscale perovskites of Example 1, particularly their thicknesses. Drop-casting a diluted toluene solution of the microscale perovskites of Example 1 on a silicon wafer afforded individual particles and small aggregates, as shown at FIG. 2C. Thicknesses of 400 nm to 600 nm for individual microscale perovskites were recorded, as shown at FIG. 2D, which was beleived to suggest that multiple corrugated layers were obtained using the new one-pot synthetic approach.

### EXAMPLE 3

### Photophysical Analysis of (EDBE)PbBr₄ Microscale Perovskites

The perovskites of Example 1 were analyzed to determine their photophysical properties.

Absorption spectrum measurements: Absorption spectrum of microscale perovskite neat thin films was conducted at room temperature through synchronous scan in an integrating sphere incorporated into the spectrofluorometer (FLS980, Edinburgh Instruments) while maintaining a 1 nm interval between the excitation and emission monochromators. The neat thin films were formed by drop-casting, as described at Example 2.

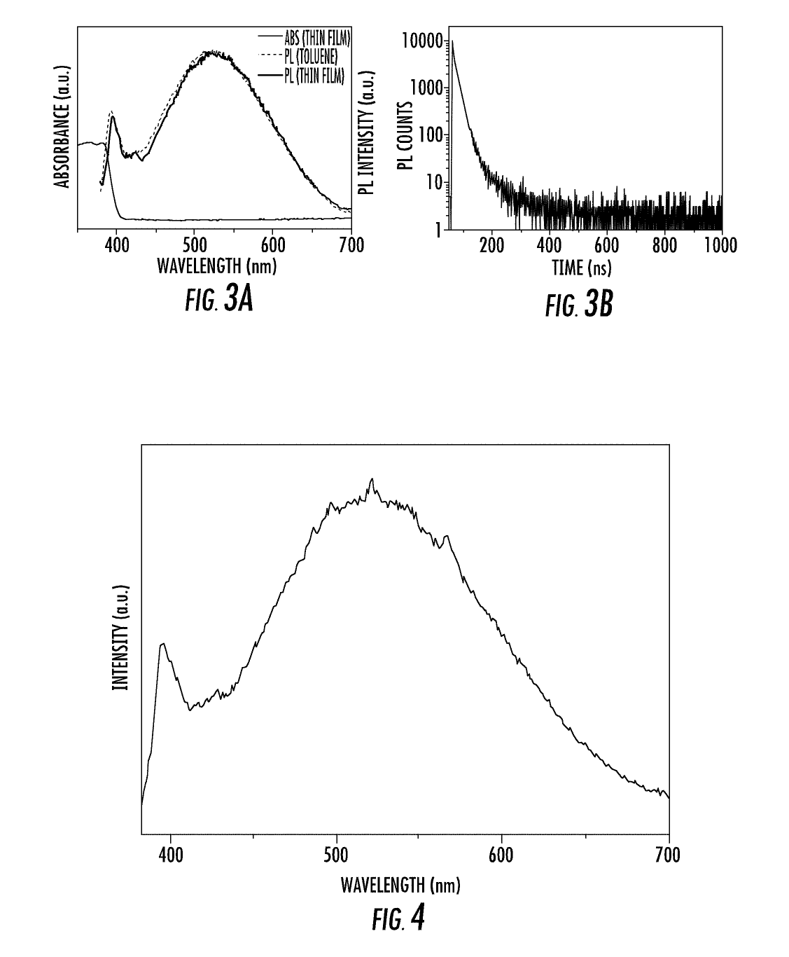

The absorption and emission spectra of the microscale perovskites of Example 1 in toluene and thin films were recorded, as shown at FIG. 3A, and summarized at Table 2.

TABLE 2
Photophysical properties of the as-prepared microcale perovskites
in toluene solution and thin film.^{a}

| | λ_{abs} | λₑₘ/FWHM | φ | τₐᵥ | kᵣ (ns⁻¹) | kₙᵣ (ns⁻¹) |
|---|---|---|---|---|---|---|
| Perovskites | (nm) | (nm) | (%)^{e} | (ns) | ×10³ | ×10³ |
| Microscale | 382 | 524/170 | 14 (18^{f}) | 21.7 | 6.45 | 39.6 |
| Crystals^{b} | | | | | | |
| Microscale | 382 | 523/171 | 12 | 20.2 | 5.94 | 43.6 |
| Crystals^{c} | | | | | | |
| Bulk Single | 371 | 573/215 | 9 | 14.0 | 6.43 | 65.0 |
| Crystals^{d} | | | | | | |

^{a}λ_{abs} is the wavelength at absorbance maximum; λₑₘ is the wavelengths at the emissio maximum; φ is the PL quantum efficiency; τₐᵥ is the PL lifetime; kᵣ and kₙᵣ are the radiative and on-radiative decay rates calculated from eqn (1) and (2);
^{b}thin film sample;
^{c}in toluene solution;
^{d}from ref.^{[13]};
^{e}error bar: ±2%;
^{f}from polystyrene thin films.

The UV-vis absorption spectrum of a neat thin film containing the microscale perovskites of Example 1 had a band edge at 405 nm. The photoluminescence spectrum of the as-prepared microscale perovskites of Example 1 in toluene showed a broad emission with a maximum at 523 nm and a large full width at half-maximum (FWHM) of 171 nm (0.79 eV) upon 360 nm excitation. There was also a less intense, but well-defined, peak at 394 nm, which was assigned to the exciton emission arising from the PbBr₄²⁻ layers. The excitation at 350-405 nm was believed to match with the emission of a typical UV-LED chip used for solid state lighting.

Not wishing to be bound by any particular theory, it was believed that the broadband emission could be attributed to the distorted excited states due to strong electron-phonon coupling in a deformable lattice, as well as inhomogeneous broadening which was believed to result from a distribution of trap states. It was believed that the strong electron-lattice coupling was likely the reason for the broad emission with a pronounced Stokes shift, due to stabilized and distorted electronic states. The neat thin film of the perovskites of Example 1 displayed an emission feature that was very similar to that of the toluene dispersion of the perovskites of Example 1.

Photoluminescence steady state studies: Steady-state photoluminescence spectra were obtained at room temperature on a Varian Cary Eclipse Fluorescence spectrophotometer. The emission spectra of the perovskites dispersed in toluene and in thin films were measured under air atmosphere (unless otherwise indicated).

Photoluminescence quantum efficiencies (PLQEs): For the photoluminescence quantum efficiency measurements, the samples were excited using light output from a housed 450 W Xe lamp passed through a single grating (1800 1/mm, 250 nm blaze) Czerny-Turner monochromator and finally a 5 nm bandwidth slit. Emission from the sample was passed through a single grating (1800 1/mm, 500 nm blaze) Czerny-Turner monochromator (5 nm bandwidth) and detected by a Peltier-cooled Hamamatsu R928 photomultiplier tube. The absolute quantum efficiencies were acquired using an integrating sphere incorporated into the FLS980 spectrofluorometer. The PLQEs reported herein were the average values from three individual experiments, with an error bar of ±2%.

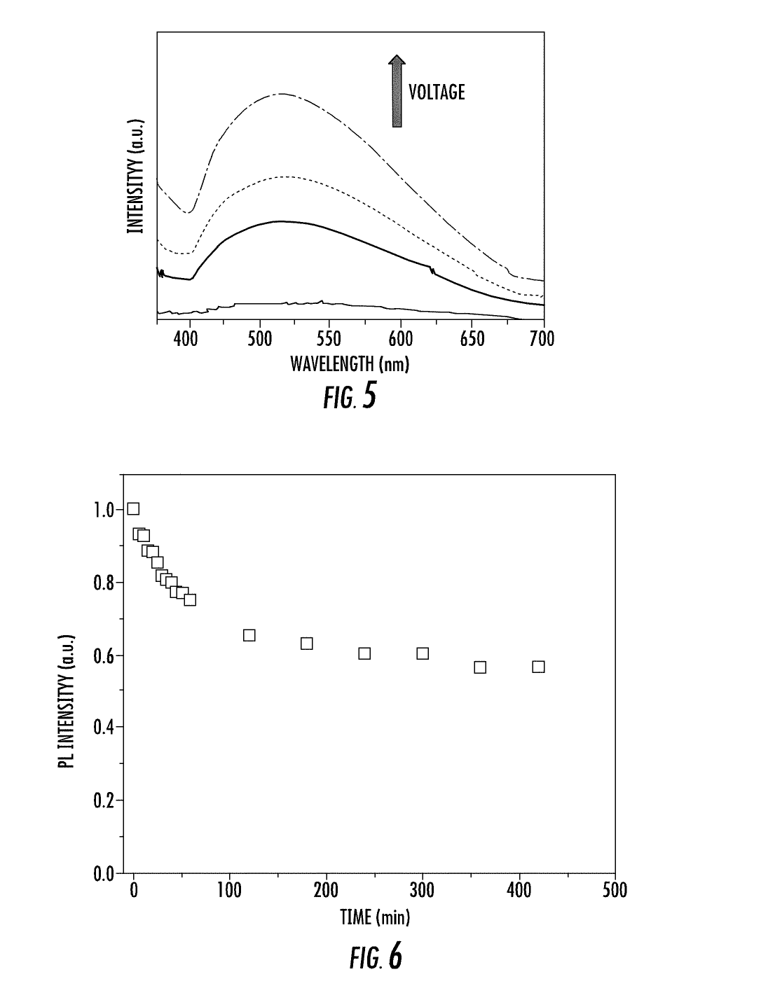

The PLQE (φ) of the microscale perovskites of Example 1 was measured at 14% for the neat thin film, 12% for the toluene solution, and 18% when embedded in a polystyrene thin film at a weight percentage of ˜10% . FIG. 4 depicts the photoluminescence spectrum of the microscale perovskites of Example 1 when embedded in polystyrene thin films at a weight percentage of 10%, λₑₓ=360 nm. The enhanced PLQE of the microscale perovskites embedded in a polymer matrix was believed to be due to reduced self-absorption and quenching.

Currently, the efficiency of a good UV-A (covering 315-400 nm) LED can be as high as 45 to 50%, leading to around 9% overall efficiency of an ideal device by using this microscale perovskite phosphor, which was comparable to compact fluorescent lamps (7-10%).

The PL lifetime (τ) of the microscale perovskites of Example 1 was determined to be 21.7 ns for the neat thin film, and 20.2 ns for the toluene solution, as shown at FIG. 3B. The similar lifetimes of the neat thin films at different emission wavelengths was believed to indicate an equilibrium on the excited state potential energy surface. The radiative (kᵣ) and non-radiative (kₙᵣ) decay rates were calculated based on the quantum efficiency (φ) and lifetime (τ) using eqn (1) and (2), and the results are listed at Table 2.

kᵣ=φ/τ (1)

kₙᵣ=(1−φ)/τ (2)

Compared to the calculated radiative (6.43×10⁻³ ns⁻¹) and non-radiative (65.0×10⁻³ ns⁻¹) decay rates of the perovskite single crystals, the microscale perovskite neat thin films had the same radiative (6.45×10⁻³ ns⁻¹) decay rate, but a much slower non-radiative (39.6×10⁻³ ns⁻¹) decay rate. This was believed to at least partially explain the higher PLQE of the as-prepared microscale perovskites compared to their bulk single crystal counterparts. This was believed to be most likely attributable to the fact that the microscale single crystals have less self-quenching effects and/or lattice defects. The radiative and non-radiative decay rates for the toluene solution of microscale perovskites were very close to those values of the neat thin film. A visual analysis of the toluene solution and neat thin film of the microscale perovskites of Example 1 under ambient light (left) and UV irradiation (right, λₑₓ =365 nm) confirmed (i) the excellent thin film formation capability of the microscale perovskites by solution processing, and (ii) the significantly enhanced white luminescence of the microscale perovskites in the solid state.

Time-resolved photoluminescence: Time-resolved emission data were collected at room temperature using an FLS980 spectrofluorometer. The dynamics of emission decay were monitored with the FLS980's time-correlated single-photon counting capability (1024 channels; 1 μs window) with data collection for 10,000 counts. Excitation was provided by an Edinburgh EPL-360 picosecond pulsed diode laser. The average lifetime was obtained from the tri-exponential decays according to equation 1:

τₐᵥₑ=Σαᵢτᵢ, i=1, 2, 3 (1)

wherein τᵢ represents the decay time and αᵢ represents the amplitude of each component.

### EXAMPLE 4

### White Light-Emitting Diodes

To demonstrate the potential application of the microscale perovskites of Example 1 as down conversion phosphors, an LED was fabricated.

The prototype solid-state lighting device of this example was prepared by dissolving the microscale perovskites of Example 1 in cyanoacrylate (Super Glue), and coating the surface of a commercial 365 nm UV-LED (Device area is ˜28.3 mm2) with the phosphor/cyanoacrylate mixture. The phosphor deposited as a thin film onto the LED surface. The device was operated at 3.0 V.

White light was emitted by the down conversion LED operated at 3.0 V. The emission spectra of the white light emitting LED collected at different voltages are shown at FIG. 5. The spectra exhibited little, if any, voltage dependence.

The white light luminance of the LED was recorded as 54.5, 68.1 and 114 cd/m² at different UV light intensities of 3, 5, and 8 mW, respectively. The performance of this device also was measured over time (up to 7 hours), and the device had at least a moderate stability in air, as shown at FIG. 6. FIG. 6 depicts the photoluminescence intensity (524 nm) of the LED device at ambient temperature over 7 hours. CIE color coordinates (0.30, 0.42) were achieved from the emission (at 3.0 V), which had a Correlated Color Temperature (CCT) of 6519 K, corresponding to a “cold” white light. These data, along with the little, if any, voltage dependence of the emission, was believed to demonstrate the high stability and good performance of the white light-emitting LED of this example.

We claim:

1. A metal halide perovskite comprising a crystal having a unit cell according to formula (I):
(A-R—B)MX₄ (I);
wherein the crystal has a layered structure;
M is a metal atom selected from Pb, Sn, Sb, Cu, Ge, Mn, Co, Bi, Ni, Zn, or Eu;
X is a halide ion selected from Cl, Br, or I; and
(A-R—B) is a ligand in which (i) A and B are independently selected from a primary ammonium cation, a secondary ammonium cation, or a tertiary ammonium cation, and (ii) R comprises a moiety of 4 to 12 atomic units, wherein each atomic unit comprises a carbon atom, a sulfur atom, or an oxygen atom.

2. The metal halide perovskite of claim 1, wherein A and B are primary ammonium cations, and the ligand is a compound of the following formula: 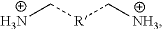
wherein R′ comprises an unbranched moiety of 2 to 10 atomic units selected independently from S, O, C, CH, or CH₂.

3. The metal halide perovskite of claim 2, wherein R′ comprises 4 to 8 atomic units selected independently from S, O, or CH₂, and the ligand is a compound of the following structure: 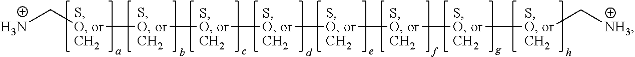
wherein a, b, c, and d are 1, and e, f, g, and h are selected independently from 0 or 1.

4. The metal halide perovskite of claim 3, wherein a, b, c, d, e, and f are 1, g and h are 0, and the ligand has the following structure:

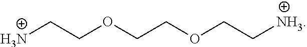

5. The metal halide perovskite of claim 3, wherein a, b, c, d, e, and f are 1, g and h are 0, and the ligand has the following structure:

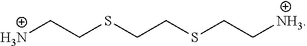

6. The metal halide perovskite of claim 1, wherein A and B are secondary ammonium cations, and the ligand is a compound of the following formula: 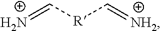
wherein R′ comprises an unbranched moiety of 2 to 10 atomic units selected independently from S, O, C, CH, or CH₂.

7. The metal halide perovskite of claim 1, wherein A and B are tertiary ammonium cations, and the ligand is a compound of the following formula: 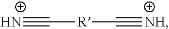
wherein R′ comprises an unbranched moiety of 2 to 10 atomic units selected independently from S, O, C, CH, or CH₂.

8. The metal halide perovskite of claim 1, wherein M is a metal atom selected from Pb, Sn, Cu, Ge, Mn, Co, Ni, or Zn.

9. The metal halide perovskite of claim 1, wherein M is Pb.

10. The metal halide perovskite of claim 1, wherein M is Pb, X is Br, and the ligand has the following structure:

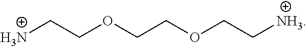

11. The metal halide perovskite of claim 1, wherein the metal halide perovskite emits white light.

12. A film comprising:
the metal halide perovskite of claim 1; and
a polymer matrix;
wherein the metal halide perovskite is dispersed in the polymer matrix, and the metal halide perovskite is present at an amount of about 1% to about 25% by weight of the film.

13. The film of claim 12, wherein the polymer matrix comprises polystyrene.

14. The film of claim 12, wherein the film emits white light, and has a PLQE of at least 15%.

15. The film of claim 12, wherein the film emits white light, and has a PLQE of about 18%.

16. A device comprising:
the metal halide perovskite of claim 1,
wherein the metal halide perovskite is a phosphor in the device, and the device emits white light.

17. The device of claim 16, wherein the metal halide perovskite is in the form of a neat film.

18. The device of claim 16, further comprising a layer of a polymer matrix, wherein the metal halide perovskite is dispersed in the polymer matrix.

19. The device of claim 16, wherein the device is a light emitting diode (LED).

20. A method of making a metal halide perovskite, the method comprising:
contacting a metal halide and an organic acid in a non-polar organic liquid; and
adding a ligand precursor to the non-polar organic liquid;
wherein the ligand precursor comprises a compound according to formula (II)
(A′-R—B′) (II),
wherein A′ and B′ are independently selected from a primary amine, a secondary amine, or a tertiary amine, and
R comprises a moiety of 4 to 12 atomic units, wherein each atomic unit comprises a carbon atom, a sulfur atom, or an oxygen atom; and
the metal halide is selected from lead(II) halide, tin(II) halide, antimony(III) halide, copper(II) halide, germanium(II) halide, manganese(II) halide, cobalt(II) halide, bismuth(III) halide, nickel(II) halide, zinc(II) halide, or europium(III) halide.

21. The method of claim 20, wherein the contacting of the metal halide and the organic acid in the non-polar organic liquid, and the adding of the ligand to the non-polar organic liquid occur an ambient temperature.

22. The method of claim 20, wherein the organic acid comprises a compound according to formula (III): 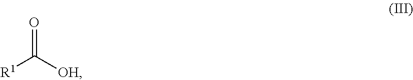
wherein R¹ is a monovalent C₅-C₂₀ hydrocarbyl.

23. The method of claim 22, wherein R₁ is a monovalent C₅-C₉ hydrocarbyl.

24. The method of claim 22, wherein R₁ is an unsubstituted, unbranched, and monovalent C₇ hydrocarbyl, and the organic acid is octanoic acid:

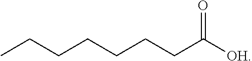

25. The method of claim 20, further comprising separating the metal halide perovskite from the non-polar organic liquid.

26. The method of claim 1, wherein the metal halide is lead(II) bromide.

27. The method of claim 20, wherein A′ and B′ are primary amines, and the ligand precursor is a compound of the following formula: 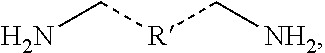
wherein R′ comprises an unbranched moiety of 2 to 10 atomic units selected independently from S, O, C, CH, or CH₂.

28. The method of claim 27, wherein the ligand precursor comprises 2,2′-(ethane-1,2-diylbis(oxy))diethanamine:

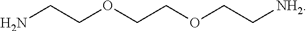

29. The metal halide perovskite of claim 1, wherein A′ and B′ are secondary amines, and the ligand precursor is a compound of the following formula: 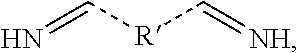
wherein R′ comprises an unbranched moiety of 2 to 10 atomic units selected independently from S, O, C, CH, or CH₂.

30. The metal halide perovskite of claim 1, wherein A′ and B′ are tertiary ammonium cations, and the ligand precursor is a compound of the following formula: 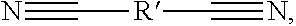
wherein R′ comprises an unbranched moiety of 2 to 10 atomic units selected independently from S, O, C, CH, or CH₂.
